(19) United States
(12) Patent Application Publication
(10) Pub. No.: US 2023/0128090 A1
(43) Pub. Date: Apr. 27, 2023

(54) MENTAL HEALTH RESPONSE SYSTEM AND METHOD

(71) Applicant: Edward Kaftarian, Calabasas, CA (US)

(72) Inventor: Edward Kaftarian, Calabasas, CA (US)

(21) Appl. No.: 17/696,765

(22) Filed: Mar. 16, 2022

Related U.S. Application Data
(60) Provisional application No. 63/161,900, filed on Mar. 16, 2021.

(51) Int. Cl. G16H 20/70; G16H 20/10; G16H 50/20; G16H 40/67; A61B 5/16

(52) U.S. Cl. CPC G16H 20/70; G16H 20/10; G16H 50/20; G16H 40/67; A61B 5/165

(57) ABSTRACT

A mental health response system and method is provided. The system and method provide guidance and support to first responders, mental health medical care providers, and others that are tasked to respond to incidents involving potential mental health crisis. The system and method inputs information regarding the incident and the patient involved in the incident and determines an incident response recommendation. The system and method provides the formulated incident response recommendation to the first responders via a mobile application. The system and method uses machine learning to determine the incident recommendation as well as follow-up medical treatment for the patient. The system and method administers the particular formulated healthcare treatment to the respective patient.

### CROSS-REFERENCE TO RELATED APPLICATIONS

This application claims priority to U.S. Provisional Application No. 63/161,900, filed Mar. 16, 2021, the entire contents of which are hereby fully incorporated herein by reference for all purposes.

### FIELD OF THE INVENTION

This invention relates to systems and methods for providing health care, including a system that provides mental health care response guidance and treatments.

### BACKGROUND

Police, paramedics, firemen and other public servants are often tasked with responding to dangerous activities and incidents. Many of these incidents involve one or more persons who may be afflicted with a mental illness or other type of development disability.

Unfortunately, it is often the case that first responders are not properly trained for dealing with those with a mental illness, and as such, may struggle during the interaction, sometimes leading to disastrous consequences.

Accordingly, there is a need for a system that provides real time mental health response guidance to those dealing with such situations. There also is a need for a system that determines and administers medical treatments based on the outcome of such situations.

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification. None of the drawings are to scale unless specifically stated otherwise.

FIG. 1 shows an overview framework of a mental health response system in accordance with exemplary embodiments hereof;

FIG. 2 shows software modules included in a mental health response system in accordance with exemplary embodiments hereof;

FIG. 3 shows a machine learning model flow in accordance with exemplary embodiments hereof;

FIG. 4 shows a machine learning model flow in accordance with exemplary embodiments hereof;

FIG. 5 shows an example workflow of actions that may be taken by a mental health response system in accordance with exemplary embodiments hereof;

FIG. 6 shows an example deployment workflow of a mental health response system in accordance with exemplary embodiments hereof;

FIG. 7 shows aspects of a mental health response system in accordance with exemplary embodiments hereof; and

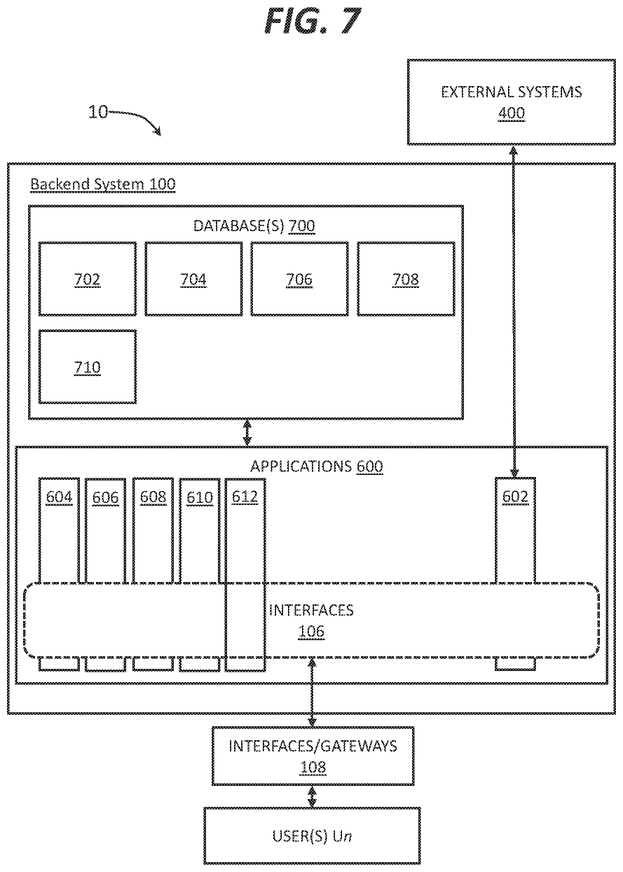

FIG. 8 depicts aspects of computing and computer devices in accordance with exemplary embodiments hereof.

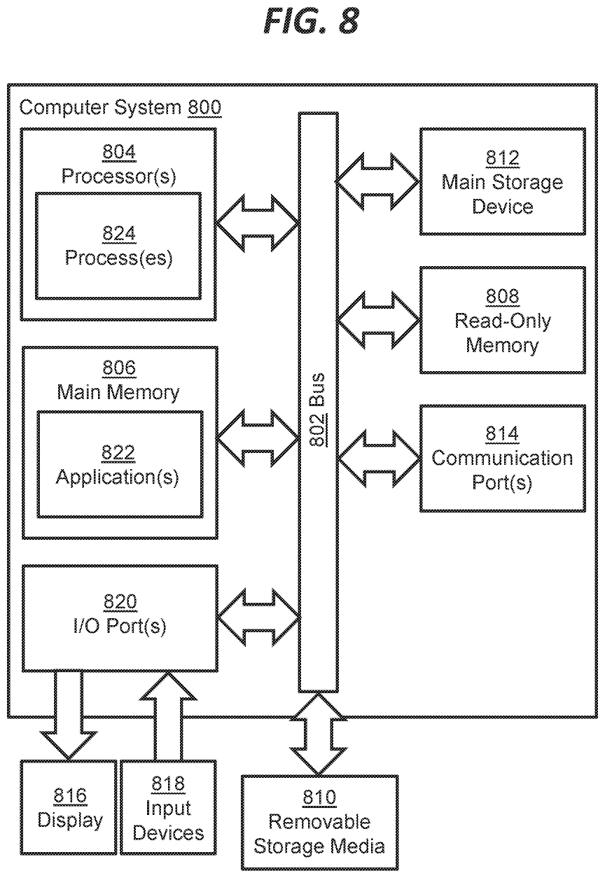

### DETAILED DESCRIPTION OF EXEMPLARY EMBODIMENTS

In general, and according to exemplary embodiments hereof, the system and method provided and described herein includes a mental health response system 10. The system 10 and method provides guidance and support to first responders, mental health medical care providers, and others that are tasked to respond to incidents involving potential mental health crisis. In some embodiments, the system 10 provides support, guidance and/or recommendations via a mobile application, a website or portal, through other types of applications and/or channels, and via any combinations thereof. In some embodiments, the system 10 may provide support to a user via online platform(s) or channels, via offline channel(s), and/or through any combination of online and/or offline platforms or channels. The system 10 also administers particular healthcare treatment plans to respective patients.

For the purposes of this specification, the term “incident” may refer to any event that involves one or more persons (e.g., an encounter between two or more persons), and typically, an event that may include dangerous, illegal, or otherwise undesirable activities. In many cases, an incident may require intervention by persons including police, firemen, paramedics, healthcare providers and/or other types of public servants. As used herein, the terms incident and encounter may be used interchangeably.

The term “response” may refer to any action or reaction taken in response to an incident. A response may be performed by first responders, public servants, healthcare providers, and/or any other appropriate individual, group of individuals and/or entities.

The term “patient” may refer to any person or persons involved in an incident that may receive a response.

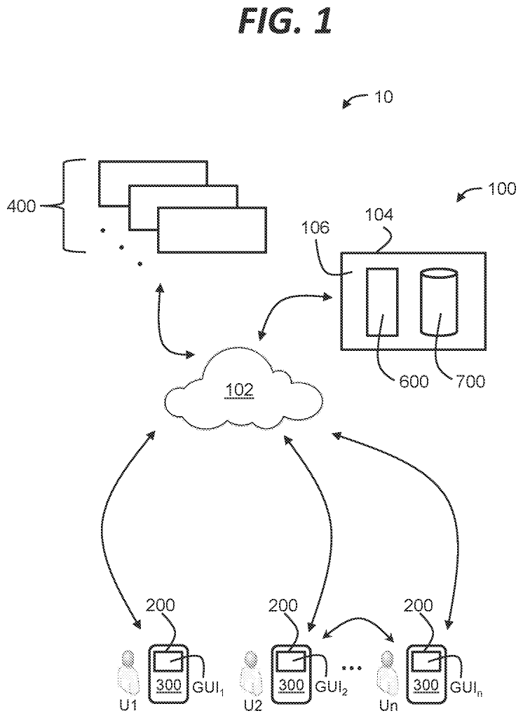

FIG. 1 shows an overview of an exemplary framework for a mental health response system 10 according to exemplary embodiments hereof. As shown, the mental health response system 10 includes a cloud platform 100 (also referred to as a backend system, backend, or controller) accessible through a network 102 such as the Internet, LAN, WAN, wireless communication systems, cellular communication systems, satellite communications systems, telephony or other types of communication systems or protocols. As will be described in other sections, the backend system 100 includes one or more servers 104 including one or more software systems 106, one or more applications 600, and one or more databases 700. The one or more software systems 106 may include operating systems, system software, web server software, social networking software, communication software, software applications, scripts, firmware, other types of software systems and any combinations thereof. The applications 600 and databases 700 will be described in other sections.

The system 10 is accessed by multiple users U0, U1, U2 . . . Un (e.g., via the network 102) using one or more applications 200 (e.g., a mobile application or “app”, a browser, and/or other type(s) of applications) running on one or more computing devices 300 (e.g., client devices such as smart phones, tablet computers, laptop computers, desktop computers, mobile media players, etc.). For the purposes of this specification, a user Un will predominantly be described as a mental health care provider and/or a first responder, however, it is understood that a user Un may include any individual or entity that may benefit from the system 10 such as, without limitation, a medical doctor, a nurse, a teacher, a professor, a therapist, a social worker, a mentor, a parent, a family member, any other type of individual and/or entity and any combinations thereof.

The system 10 also may interface with various external entities and/or systems 400 such as police or fire department dispatchers, hospitals, medical offices, databases and/or systems that include information regarding police records, healthcare records, driver information, legal information, and other types of systems. The system 10 also may interface with mental healthcare providers (e.g., psychiatrists, psychologists, therapists, social workers, medical doctors, etc.), public servants (e.g., police officers, firemen, paramedics, etc.), and/or other types of external entities or systems 400. In this way, the system 10 may retrieve information from and share information with the external systems 400 (preferably in real time) during use of the system 10 and as the system 10 performs its functionalities.

In some embodiments, the mental health response system 10 may perform at least some of the following activities (in real time) prior to or at the onset of a response, during a response, and/or after a response:
1. Acquire data, information and/or other types of assets relating to an incident and a response, and subsequently dispatch an appropriate user Un to the scene of the incident to facilitate a resolution of the encounter. This may include acquiring any information relating to the patient P, the user Un, other responders, the encounter (e.g., type of encounter, location, extenuating circumstances, etc.), and/or other entities (e.g., other healthcare providers, etc.). The information may be acquired from any source including but not limited to the mobile application 200 (e.g., the user Un), the external systems 400, or other sources.
2. Formulate a response to the incident based at least in part on the information acquired in (1) above. The response may include recommended actions that may be taken by a user Un, a step-by-step process that may be performed, other types of guidance regarding the encounter and the patient, and other types of information. Note that the system 10 may modify the recommended response (preferably in real time) as the incident proceeds and as additional information may be acquired. In this way, the recommended response may be fluid and evolving.
3. Deliver the recommended response and guidance to a user Un. The response may be provided via the mobile application 200 on the user's device 300, or through other means. Depending on the recommended response and the guidance, the mobile application 200 may provide one or more graphical user interfaces (GUIs) that may include wizards, multimedia elements (graphics, video, audio, etc.), step-by-step guidance, interactive elements and other aspects that may provide the guidance to the user Un.
4. Provide communication channels or links between the user Un and/or the patient P, and any desired external systems 400 (e.g., healthcare providers). Such communication links may include video conferencing, voice, text, email, chat, any other types of communications and any combinations thereof. For example, the system 10 may deem it necessary for a user Un to interface with a psychiatrist or other healthcare provider during his/her interaction with the patient P, and this interaction may be facilitated via the mobile application 200 running on the user's mobile device 300.
5. Provide training to the user Un regarding strategies to best navigate and support different types of incidents with different types of patients P. This may include storing information regarding past incidents and providing them as simulations for training purposes. Other types of training also may be provided such as tutorials, classes, live instruction (e.g., via multimedia conferencing), and other types of training.
6. Administer a particular healthcare treatment plan to the patient P specifically based at least in part on the incident, the patient P, the incident response, and the incident's outcome.
7. Other types of functionalities as described herein.

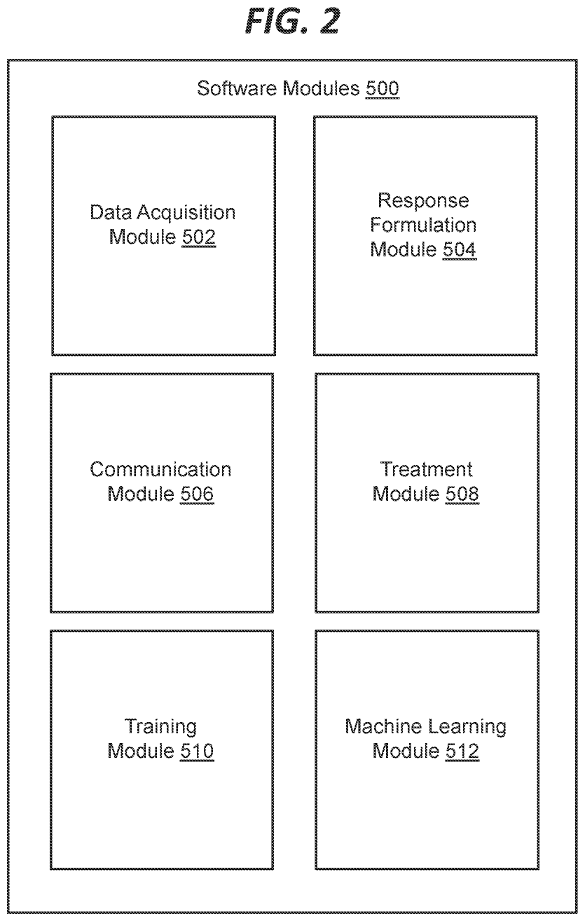

In some embodiments as shown in FIG. 2, the system 10 includes one or more modules 500, with each module 500 providing one or more functionalities of the system 10.

For example, in some embodiments the system 10 may include the following modules 500:
1. Data Acquisition Module 502;
2. Response Formulation Module 504;
4. Communication Module 506;
5. Treatment Module 508;
6. Training Module 510; and
7. Machine Learning Module 512.

It is understood that the system 10 may include additional modules 500 and/or not all of the modules 500 listed above.

### Data Acquisition Module 502

In some embodiments, the data acquisition module 502 receives data, information, and other types of assets from other sources (e.g., from external systems 400) and inputs the data, information, and other types of assets into the system 10. The data acquisition may occur at any time, including prior to or at the onset of the response, during the response, and/or after the response.

In some embodiments, the module 502 interacts with an external system 400 such as a police dispatch system to acquire information regarding an incident and a potential response. For example, the police dispatch system may convey to the module 502 the type of incident, specific details surrounding the incident, the persons involved with the incident, the location of the incident, the current status of the incident, the emergency level of the incident, whether the incident is violent or nonviolent, the police officers on the scene, and other pertinent information. In another example, the module 502 may interact with a police records system to receive information regarding the patient's police record, a medical records system to receive information regarding the patient's medical history, or the DMV to receive information regarding the patient's driving record. It is understood that the system 10 may interface with other external systems 400 (e.g., fire department dispatch system, paramedic dispatch system, etc.) to acquire information, as necessary.

In other embodiments, the module 502 receives data and other assets regarding the incident and potential response from input mechanisms such as the application 200, web portals, websites, other types of applications and any combinations thereof. For example, the user Un may input relevant information using one or more GUIs provided by the application 200. In another example, a dispatch operator may input information regarding the incident into a website or portal that may then be uploaded to the system 10 via the data acquisition module 502.

Once the data, information or other assets are received, the module 502 may store the data, information, and other assets to one or more databases 700 of the system 10.

In some embodiments, the data acquisition module 502 continues to receive and/or acquire new information as the response proceeds and provides the new information to the system 10 (preferably in real time). In this way, the system 10 may continually update its recommendations based on the new information (preferably in real time).

It is understood that the data acquisition module 502 may acquire any type of information from any source for use with the system 10, and that the system 10 is not limited in any way by the source(s) and/or types of information that the module 502 may acquire.

### Response Formulation Module 504

In some embodiments, the response formulation module 504 uses at least some of the information acquired by the data acquisition module 502 (and/or received from an external entity 400 as described in other sections) to formulate a recommended response to the incident. For example, the response formulation module 504 may utilize information that indicates that the patient P has an anger management problem and a tendency to resort to violence and may formulate an appropriate response to deescalate the situation to help the patient P remain calm. At the same time, the module 504 may warn the user Un of this fact so that the user Un may take the proper safety precautions. In another example, the module 504 may utilize information that indicates that the patient has suicidal tendencies and may formulate an appropriate response to act quickly and to prevent the patient from hurting themselves. It is understood that the examples above are meant for demonstration and that the module 504 may utilize any pertinent information to formulate any appropriate recommended actions.

In some embodiments, the response formulation module 504 continually updates its recommendations and/or guidance as it receives new information from the data acquisition module 502. For example, the user Un may enter information into the mobile application 200 during his/her interaction with the patient P indicating that the patient seems to be under the influence of drugs and/or alcohol (and therefore may potentially act irrationally), and the response formulation module 504 may alter its guidance to the user Un accordingly.

In some embodiments, the response formulation module 504 provides its recommendations and guidance to the user Un via the mobile application 200. The recommendations and guidance also are stored by the system 10 into a database 700. For example, the mobile application 200 may present one or more GUIs to the user Un that show decision trees of possible questions that the user Un may ask the patient P, and the recommended follow up responses that the user Un may take or provide given a particular response from the patient P. In another example, the response formulation module may provide one or more multimedia files (e.g., videos, animations, wizards, etc.) to the user Un providing step-by-step actions that the user Un may take. It is understood that the response formulation module 504 may provide any type of guidance or information to the user Un using any means available, and that the system 10 is not limited in any way by the information or by the way that the information may be provided.

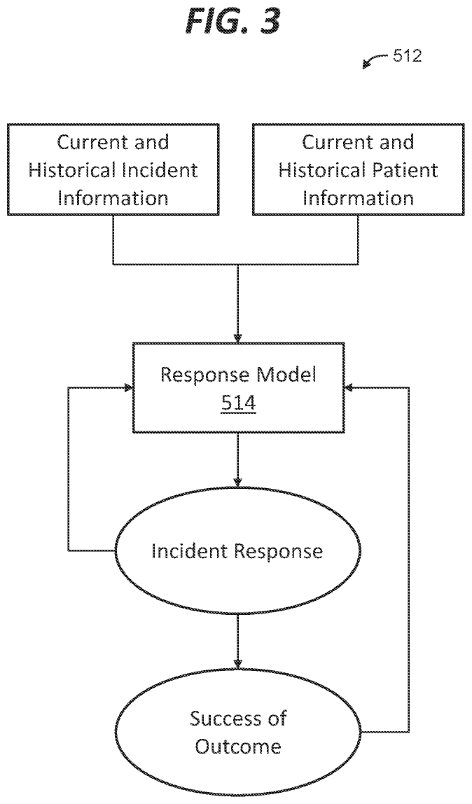

In some embodiments, as shown in FIG. 3, the response formulation module 504 employs artificial intelligence (AI) using current and historical incident data and current and historical patient information to predict possible incident outcomes and to formulate a recommended incident response. For example, in some embodiments, the response formulation module 504 employs the machine learning module 512 which includes neural networks, deep learning, logic tree decisions, fuzzy logic, smart agent profiling, and case reasoning to predict possible incident outcomes and to formulate a recommended incident response.

As shown in FIG. 3, the response model 514 of the machine learning module 512 may be trained using the current incident information, historical incident information, current patient information, and historical patient information. Additionally, the response model 514 may be further trained by using current incident response information, historical incident response information, and information relating to the success (or lack of success) of each incident response.

As shown in FIG. 3, once the response model 514 is adequately trained, the machine learning module 512 uses the response model 514 to predict a desired incident outcome (e.g., safe de-escalation of the incident) and to determine an incident response with the highest level of confidence to achieve the desired incident outcome. The response formulation module 504 may receive the recommended incident response from the machine learning module 512 and present it to the user Un via the application 200.

In some embodiments, as shown in FIG. 3, the machine learning module 512 also may grade the success (or lack of success) of the administered incident response and may use this information to further train the response model 514. For example, if the recommended incident response was successful in leading to a safe de-escalation of the incident, the response model 514 may be further trained as such. However, if the recommended incident response was not successful in leading to a safe de-escalation of the incident, then the response model 514 may be further trained given this information.

In some embodiments, the response formulation module 504 may recommend contacting a mental healthcare provider (e.g., a psychiatrist, psychologist, therapist, etc.) in real time during the user's interaction with the patient P so that the healthcare provider may assist in the response. This will be described below.

### Communication Module 506

In some embodiments, when the system 10 (e.g., the response formulation module 504 as described in other sections) deems it beneficial to include real time communications with one or more external entities 400 (e.g., a psychiatrist, psychologist, therapist, etc.) during the response, the communications module 506 may automatically contact the desired entity 400 and provide a channel of communication between the user Un and the entity 400. Alternatively, in some embodiments, the user Un may initiate the communications with the external entity 400 with or without the recommendation of the module 504 as he/she may see fit.

In some embodiments, the communication module 506 may provide a communication channel between the user Un and the external entity 400 via one or more GUIs on the mobile application 200. In one example, the module 506 may provide a live video conferencing channel so that the user Un and/or the patient P may interface with the entity 400 in real time via video. In another example, the module 506 may provide a voice channel, a text channel, a chat channel, and/or other types of communications to interface with an external entity 400.

In some embodiments, an external entity 400 (e.g., an external entity 400 communicating with the patient P and/or the user Un in real time during the incident) may provide recommendations regarding one or more courses of action that the system 10 (and possibly the user Un) should take. For example, the external entity 400 may diagnose the patient P during the interaction and provide a suggested treatment plan to the system 10 (e.g., via a GUI of the application 200). This information may be received by the system 10 and stored into a database 700 for real time and/or future use (e.g., for use by the response formulation module 504 and/or the treatment module 508 as described in other sections).

### Treatment Module 508

In some embodiments, the system 10 includes a treatment module 508 that administers one or more particular mental healthcare treatments or programs to the patient P specifically based at least in part on the data collected and analyzed by the system 10 regarding the incident and/or the patient P including the patient's P's background. In this way, the system 10 effects a particular treatment or prophylaxis for the patient P for a disorder, disease, or medical condition.

In some embodiments, the treatment module 508 processes information received by the data acquisition module 502, by the response formulation module 504, and/or by the communications module 506 (e.g., via an external entity 400) and determines one or more mental health disorders that the patient P may be suffering from. Next, the treatment module 508 uses the aforementioned information to determine one or more treatment plans to administer to the patient P. Subsequently, the system 10 administers the treatment plan to the patient P as described herein.

For example, in some embodiments, the system 10 may determine, given the acquired information, that the patient P suffers from depression and requires a particular type of therapy (e.g., a therapy treatment for depression). For instance, the treatment module 508 may determine that the patient P is suicidal and administers cognitive behavioral therapy (CBT), and/or dialectic behavior therapy (DBT) to the patient P.

In another example, in some embodiments, the treatment module 508 may determine that the patient is addicted to one or more substances (e.g., opioids, alcohol, etc.), and administers a substance abuse treatment and/or addiction treatment to the patient P including detox, rehab, cognitive behavioral therapy (CBT), rational emotive behavioral therapy (REBT), contingency Management (CM), and/or 12-step facilitation therapy.

In another example, the treatment module 508 may determine that the patient P suffers from depression and administers psychotherapy, cognitive behavioral therapy (CBT), dialectic behavior therapy (DBT), psychodynamic therapy, and/or interpersonal therapy to the patient P.

In another example, the treatment module 508 may determine that the patient P suffers from anxiety and administers psychotherapy, cognitive behavioral therapy (CBT), dialectic behavior therapy (DBT), psychodynamic therapy, and/or interpersonal therapy to the patient P.

It is understood that the system 10 matches its diagnosis of the patient P with the proper type of therapy depending on the particular circumstances regarding the incident and the patient's P's prior history (including prior incidents, medical history, criminal history, etc.), and subsequently administers the treatment to the patient P.

In some embodiments, depending on the data that the system 10 collects during and/or after the incident, the system 10 administers a particular therapy such as Accelerated Experiential Dynamic Psychotherapy (AEDP), Acceptance and Commitment Therapy, Adlerian Therapy, Affirmative Psychotherapy, Anger Management Therapy, Animal-Assisted Therapy, Applied Behavior Analysis, Art Therapy, Attachment-Based Therapy, Bibliotherapy, Biofeedback, Brain Stimulation Therapy, Christian Counseling, Client-Centered Therapy (Person-Centered Therapy, PCT, CCT or Rogerian Therapy), Coaching, Cognitive Behavioral Therapy, Cognitive Processing Therapy, Cognitive Stimulation Therapy, Collaborative Therapy, Compassion-Focused Therapy, Conflict Resolution Therapy, Contemplative Psychotherapy, Core Process Psychotherapy, Culturally Sensitive Therapy, Dance Therapy, Dialectical Behavior Therapy, Eclectic Therapy, Ego State Therapy, Emotionally Focused Therapy, Equine-Assisted Therapy, Existential Therapy, Experiential Therapy, Exposure and Response Prevention, Expressive Arts Therapy, Eye Movement Desensitization and Reprocessing Therapy. Family Systems Therapy Feminist Therapy, Forensic Therapy, Gender Aware Therapy, Gestalt Therapy, Holistic Psychotherapy, Human Givens Therapy, Humanistic Therapy, Hypnotherapy, Imago Relationship Therapy, Integrative Therapy, Intensive Short-Term Dynamic Psychotherapy, Internal Family Systems Therapy, Interpersonal Psychotherapy, Journey Therapy, Jungian Therapy, Logotherapy, Marriage and Family Therapy, Mentalization-Based Therapy, Mindfulness-Based Cognitive Therapy, Motivational Enhancement Therapy, Motivational Interviewing, Multicultural Therapy, Music Therapy, Narrative Therapy, Neuro-Linguistic Programming Therapy, Neurofeedback, Parent-Child Interaction Therapy (PCIT), Person-Centered Therapy, Play Therapy, Positive Psychology, Prolonged Exposure Therapy, Psychoanalytic Therapy, Psychodynamic Therapy, Psychological Testing and Evaluation, Rational Emotive Behavior Therapy, Reality Therapy, Redecision Therapy, Regression Therapy, Relational Therapy, Religious Therapy, Sand-play Therapy, Schema Therapy, Social Recovery Therapy, Solution-Focused Brief Therapy, Somatic Therapy, Strength-Based Therapy, Structural Family Therapy, Symbolic Modelling, The Gottman Method, Therapeutic Intervention, Transpersonal Therapy, Trauma-Focused Cognitive Behavior Therapy, Addiction Counseling, Divorce Counseling, Erectile Dysfunction Sex Therapy, Grief Counseling, Postpartum Counseling, Sex Therapy, Therapy for Infertility, Therapy for Infidelity, Therapy for Miscarriages, Therapy for Pregnancy, and other types of therapies.

In some embodiments, the system 10 administers the determined therapy (treatment) to the patient P by notifying the patient P of the required treatment, by scheduling the required treatment with a properly credentialed healthcare provider (e.g., the psychiatrist 400 that interacted with the patient P during the incident, a detox and/or rehab facility, etc.), by issuing an order (e.g., a court order or other type of order) to the patient P requiring his/her participation in the treatment, by confirming at the time/date of the treatment that the patient P is in attendance at the required treatment, by confirming at the time/date that the patient P is in fact receiving the treatment (e.g., from the healthcare provider 400), confirming at the time/date that the patient P completed the treatment, and other administrative actions. In some embodiments, the various confirmations may be performed via a GUI on the application 200 by the healthcare provider in real time at the time/date of the treatment.

In some embodiments, the system 10 may provide the required treatment via multimedia files presented by a GUI on the application 200 (e.g., video, audio, animations, presentations, etc.) with and/or without the involvement of an external entity 400.

In another example, in some embodiments, the system 10 may determine, given the acquired information, that the patient P needs a particular type of medication. For instance, the treatment module 508 may determine that the patient P is suffering from depression and administers a selective serotonin reuptake inhibitor (SSRI), such as citalopram (Celexa®), escitalopram oxalate (Lexapro®), fluoxetine (Prozac®) fluvoxamine (Luvox®) paroxetine HCI (Paxil®) and sertraline (Zoloft®), a selective serotonin and norepinephrine inhibitor (SNRIs), such as desvenlafaxine (Khedezla®), desvenlafaxine succinate (Pristiq®), duloxetine (Cymbalta®), levomilnacipran (Fetzima®) and venlafaxine (Effexor®), a novel serotonergic drug such as vortioxetine (Trentellix®-formerly called Brintellix®) or vilazodone (Viibryd®), a tricyclic antidepressant, such as amitriptyline (Elavil®), imipramine (Tofranil®), nortriptyline (Pamelor®), and doxepin (Sinequan®), a drug that affects mainly dopamine and norepinephrine such as bupropion (Wellbutrin®), a monoamine oxidase inhibitor (MAOIs), such as isocarboxazid (Marplan®), phenetzine (Nardil®), Selegiline (EMSAM®), and tranylcypromine (Parnate®), a tetracyclic antidepressant that is noradrenergic and specific serotonergic antidepressants (NaSSAs), such as mirtazapine (Remeron®), and L-methylfolate (Deplin®).

In another example, in some embodiments, the treatment module 508 may determine that the patient has a substance abuse disorder (e.g., is addicted to one or more substances such as opioids, alcohol, etc.), and administers a drug withdrawal and/or detox medication to help alleviate withdrawal symptoms during the detox period. For example, during the first stage of detox, acute withdrawal, the treatment module 508 administers benzodiazepines, antidepressants, and/or clonidine. Next, during the post-acute withdrawal period, the treatment module 508 administers a post-acute withdrawal medication depending on the type of addiction. For example, in some embodiments, if the treatment module 508 determines that the patient P is suffering from alcoholism, the treatment module 508 administers naltrexone (Vivitrol®), acamprosate (Campral®), and/or disulfiram (Antabuse®).

In another example, if the treatment module 508 determines that the patient P is suffering from opiate addition, the treatment module 508 administers methadone, buprenorphine (Suboxone®), and/or Naltrexone.

In some embodiments, the system 10 administers the determined medication to the patient P by notifying the patient P of the required medication, by sending a workorder to the healthcare provider (e.g., the psychiatrist 400 that interacted with the patient P during the incident) notifying him/her of the determined medicinal treatment plan that is to be prescribed to the patient P, by prescribing the required medication to

patient P via the healthcare provider, by issuing an order (e.g., a court order or other type of order) to the patient P requiring him/her to take the medication and confirming that the patient P has indeed taken the medication (e.g., by scheduling follow-up appointments between the patient P and the provider and confirming that the patient P has attended the appointments). In some embodiments, the various confirmations may be performed via a GUI on the application 200 by the healthcare provider in real time at the time/date of the treatment.

In some embodiments, as shown in FIG. 3, the treatment module 508 employs artificial intelligence (AI) using current and historical incident data, current and historical patient information, and current and historical incident response information to formulate a recommended treatment. For example, in some embodiments, the treatment module 504 employs the machine learning module 512 which includes neural networks, deep learning, logic tree decisions, fuzzy logic, smart agent profiling, and case reasoning to predict possible incident outcomes and to formulate a recommended treatment.

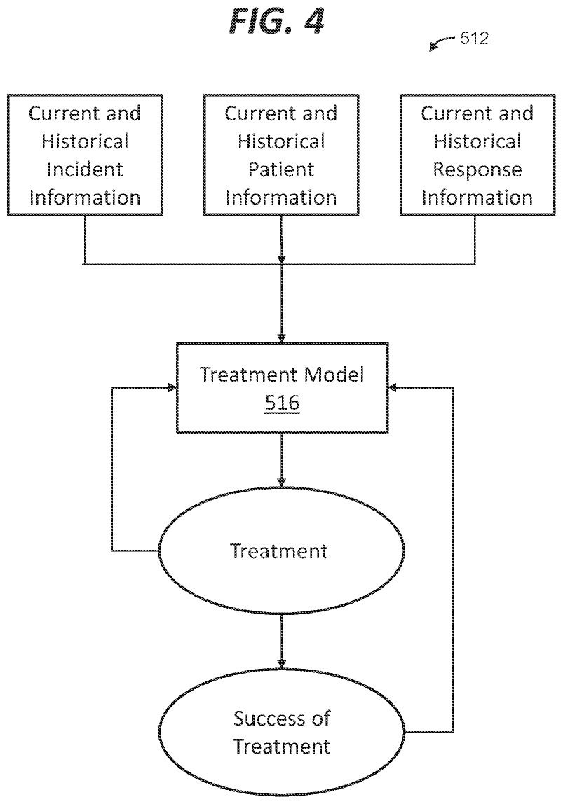

As shown in FIG. 4, the treatment model 516 of the machine learning module 512 may be trained using the current incident information, historical incident information, current patient information, historical patient information, current incident response information, and historical incident response information. Additionally, the response model 514 may be further trained by using current treatment information, historical treatment information, and information relating to the success (or lack of success) of each administered treatment.

As shown in FIG. 4, once the response model 514 is adequately trained, the machine learning module 512 uses the treatment model 516 to predict a successful treatment outcome (e.g., a healthcare treatment to address the mental health disorder of the patient P) and to determine a treatment with the highest level of confidence to achieve the desired treatment outcome. The response formulation module 504 may receive the recommended treatment from the machine learning module 512, present it to the user Un via the application 200, and administer the treatment to the patient P (e.g., using the treatment module 508) as described in other sections.

In some embodiments, as shown in FIG. 4, the machine learning module 512 also may grade the success (or lack of success) of the treatment given the administration of the treatment and may use this information to further train the treatment model 516. For example, if the administered treatment was successful in leading to a reduction of symptoms of the patient's P's mental health disorder, the treatment model 516 may be further trained as such. However, if the administered treatment was not successful in providing relief to the patient P, then the treatment model 516 may be further trained given this information.

It can be seen in FIGS. 3 and 4 that the treatment model 516 in FIG. 4 is trained in part by outcomes provided by the response model 514 in FIG. 3, thereby adding an additional layer of complexity to the actions performed by the system 10 that would be difficult and challenging for persons knowledgeable in the art to perform mentally due to their computational complexity.

In this way, the system 10 effects a particular treatment or prophylaxis for the particular disorder, disease, or medical condition (e.g., mental health disorder or condition) that the patient P may suffer.

### Training Module 510

In some embodiments, the system 10 includes a training module 510 that may train or otherwise teach users Un different strategies and actions that may be taken during specific types of incidents. It may be preferred that the training take place using simulated incidents, but this may not be required.

In some embodiments, the system 10 stores (into its database(s) 700) information pertaining to one or more incidents. The information may include the data defining the type of incident, the type(s) of patient(s) involved with the incident, the type(s) of healthcare providers involved in the incident, actions that may have been taken by the user Un, by the patient P, by the police officers at the scene, by the external entity 400 (e.g., mental healthcare provider) and other information pertaining to the circumstances of the incident. The system 10 also may store the outcome of the incident, information regarding the determined treatment, information regarding the administration of the treatment, and the results of the administration of the treatment (e.g., successes and/or challenges faced).

In some embodiments, the training module 510 may utilize the stored historical information to develop one or more simulated incidents, and in some embodiments, may present the simulated incidents to a user-in-training as a training exercise. During a simulation, the system 10 may present to the user Un the circumstances surrounding the simulated incident and may guide the user Un through the simulation so that the user Un may experience the challenges that the incident presented.

In other embodiments, the training module 510 enables users Un to create and run their own simulations, whether the user-created simulations include information from prior incidents, or not.

In other embodiments, the training module 510 may use historical information from prior incidents to create and provide training tutorials, training manuals, multimedia training materials (e.g., videos), and/or any other type(s) of training assets.

It is understood that the training module 510 may utilize any information from prior incidents or otherwise to provide training materials of any kind, and that the system 10 is not limited in any way by the types of training materials that the training module 510 may create and/or provide.

In any of the modules 500 and/or embodiments described herein, the system 10 also may employ industry standards, protocols, techniques, procedures, treatments, and/or other regulations as required by law, as accepted by industry professionals or otherwise.

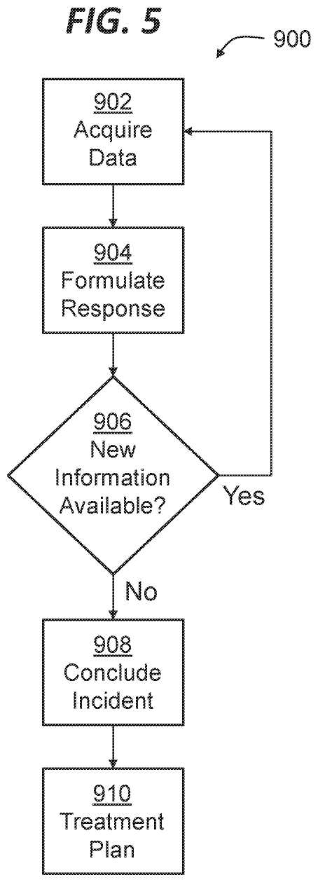

In some embodiments as shown in FIG. 5, a possible workflow 900 of the system 10 is depicted.

At 902, the data acquisition module 502 may acquire data as described herein. Then, at 904, the response formulation module 504 may utilize the acquired data to formulate a first recommended response.

As the incident/response continues, if the incident evolves or otherwise changes such that new information is available (at 906), the system 10 may employ the data acquisition module 502 to acquire the new information (again at 902). Then, given the new information, the response formulation module 504 may modify and provide to the user Un a newly updated response plan (again at 904).

Once there is no new information and the incident concludes (at 908), the treatment module 508 may administer an appropriate mental healthcare treatment plan at 910.

It is understood that the workflow 900 is meant for demonstration and that the system 10 may follow other workflows as appropriate for specific incidents and/or responses.

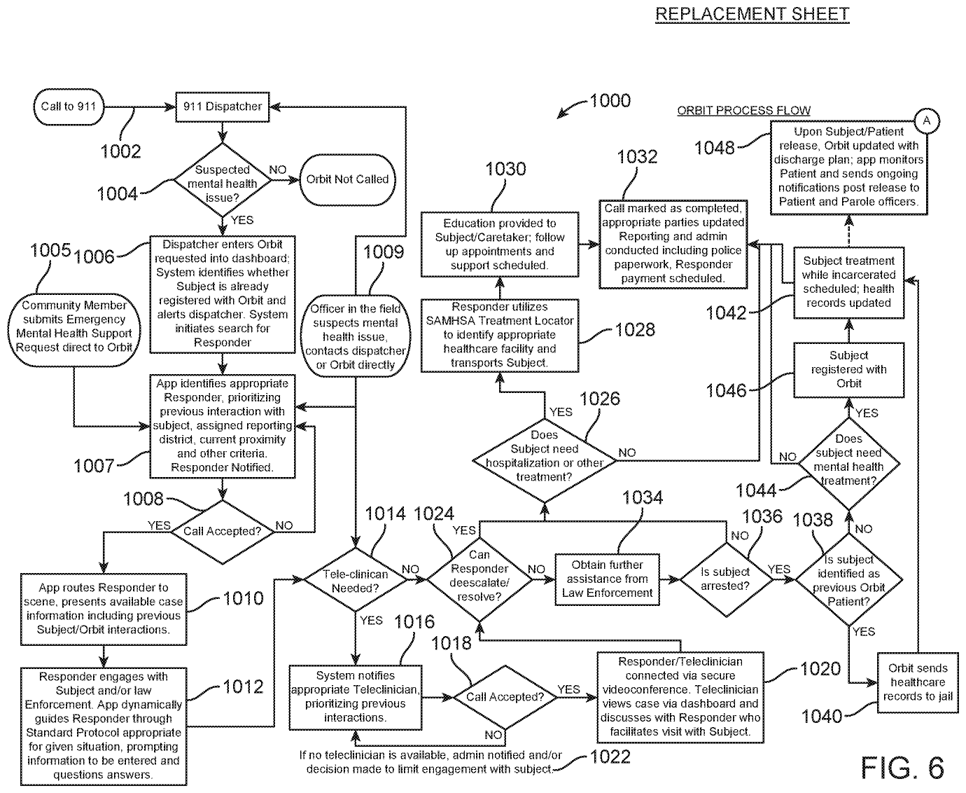

FIG. 6 depicts an example deployment workflow 1000 of the system 10.

At 1002, an emergency 911 call of an incident is received at the 911 dispatch. If it is deemed at 1004 that the incident involves a suspected mental health issue, the dispatcher enters a request for the system 10 at 1006 at which time the system 10 determines if the incident involves one or more persons (patients P) already registered with the system 10. This information is provided to the dispatcher and to other users Un of the system 10, and the system 10 initiates a search for a qualified responder Un. Note also that a community member may submit an emergency mental healthcare support request to the system 10 directly at 1005 and/or a law enforcement officer may submit a similar request to the system 10 at 1009, and in doing so may or may not bypass the 911 dispatch service.

At 1007, the system 10 identifies one or more appropriate responders Un, and prioritizes the identified responders Un using one or more criteria, including without limitation, previous interaction with the patient P, the assigned reporting district, current proximity to the location of the incident, and other criteria. Once identified, the system 10 initiates contact with the responder Un (e.g., by calling the responder Un).

If the identified responder Un does not accept the call from the system 10 at 1008, the workflow reverts to 1007 and the next prioritized responder Un is identified, etc.

If the identified responder Un accepts the call at 1008, the system 10 routes the responder Un to the scene of the incident at 1010 while providing the responder Un with the pertinent information including history of any prior interaction(s), subject matter of the incident, etc. The responder Un arrives at the scene at 1012 and interacts with the patient P. During this process, the system 10 may dynamically guide the responder Un through standard protocols determined appropriate for the given incident (e.g., via the mobile app 200 through one or more GUIs). The system 10 also may prompt the responder Un to provide feedback information received from the patient P (or otherwise) to the system 10 that the system 10 may upload and store to one or more databases 700 on the backend 100.

Then, at 1014, if the system 10 deems that real time communication with a healthcare provider (an external entity 400 such as a teleclinician) would be beneficial, the system 10 may attempt to establish contact with a healthcare provider at 1016. The healthcare provider may be chosen using one or more criteria including for example, whether or not the provider has had prior contact and/or interaction with the patient P. Conversely, if the system 10 deems that a teleclinician is not needed, the workflow may move directly to 1024.

If the system 10 is able to establish contact with the teleclinician at 1018, the teleclinician is connected to the system 10 via a secure videoconferencing link at 1020 and may interact with the user Un, the patient P, law officers and other entities as appropriate. The system 10 also may provide the teleclinician with a GUI dashboard (e.g., via a mobile app 200) that provides background information regarding the incident, the patient P, and other pertinent information regarding the case. If, however, the system 10 is not able to establish contact with a suitable healthcare provider, the system 10 may or may not instruct the responder Un to limit his/her engagement with the patient P at 1022.

Then, if the user Un and/or the external entity 400 (e.g., the teleclinician) is able to deescalate the incident at 1024, the incident is concluded, and the patient P is examined for medical attention at 1026. If the patient P requires medical attention, the system 10 may utilize a healthcare facility locator (such as SAMHSA Treatment Locator) at 1028 to identify an appropriate facility for the patient P, and to transport the patient P to the facility as required. Once at the facility at 1030, treatment may be provided to the patient P, and educational information may be provided to both the patient P and the caregiver Un. Follow-up treatment plans, appointments and/or other types of support also may be scheduled.

Following 1030, and/or if it is determined that the patient P does not require medical attention at 1026, the call may be marked as completed at 1032. The system 10 may then update the appropriate parties regarding the incident and the outcome and perform any appropriate administrational work, as necessary. This may include providing administrative reports, healthcare reports, police reports, billing, payment to responder Un and/or the teleclinician, and other types of work. The system 10 also may save all relevant data to its databases 700, and perform other tasks, as necessary.

Returning to 1024, if the responder Un and/or the teleclinician is unable to deescalate the incident, further law enforcement assistance may be obtained at 1034. If this assistance does not result in the arrest or detainment of the patient P at 1036, then the workflow may move to 1026 as described above.

If, however, the patient P is arrested and taken into custody at 1036 and the system 10 identifies the patient P as a previous system patient P at 1038, the system 10 may provide the appropriate law enforcement entities (e.g., the local police station or jail) any appropriate records pertaining the patient P (e.g., healthcare records) at 1040. The patient P may then be treated while incarcerated at 1042 and the system records may be updated accordingly.

If, however, the patient P is not identified as a prior system patient P at 1038, the patient P may be assessed for needing mental healthcare treatment at 1044. If it is determined that the patent P does not require mental healthcare treatment at 1044, the workflow may move to 1032 as described above. If it is determined that the patient P does in fact require mental healthcare treatment at 1044, the patient P may be registered with the system 10 at 1046 and the workflow may move to 1042 where the patient P may receive treatment while incarcerated.

It is understood that the workflow 1000 described above is meant for demonstration and that the system 10 may follow other workflows during deployment as required.

As mentioned above, at 1012 the system 10 may dynamically guide the responder Un through standard protocols determined appropriate for the given incident (e.g., via the mobile app 200 through one or more GUIs). In some embodiments, this may include, without limitation, asking a series of questions, and depending on the answers received, asking subsequent follow-up questions (e.g., predetermined and/or determined in real time using the response formulation module 504 and/or the machine learning module 512). For example, the system 10 may ask “does the patient P appear to be talking to himself/herself and/or hallucinating?”. If the responder Un answers “yes”, the system 10 may use this answer to determine and provide a follow-up question such as “is the voice familiar to them or unfamiliar?”. If the responder Un answers “unfamiliar”, the system 10 may ask a follow-up question such as “is the voice telling him/her to kill themselves?”. The system 10 may again ask another follow-up question and the process may continue until a resolution is reached or otherwise. As described, the answer to each question may spawn a new question that asks for additional information specific to the incident. The system 10 may store the questions and responses into a database 700 on the backend 100 and the data may be used for future diagnosis and/or reporting. It is understood that the questions and answers provided above in this example are meant for demonstration and that the system 10 may ask any series of questions as appropriate.

In some embodiments, the system 10 also may provide support to incarcerated or otherwise detained patients P upon their release from prison or other types of detainment. In some embodiments, a patient P, upon his/her release, may register with the system 10 and install the mobile app 200 onto his/her device 300. This may be a mandatory condition upon being paroled or voluntary.

In some embodiments, the system 10 may provide at least some of the following functionalities to these patients P (or others) at 1044.
1. The system 10 administers the particular treatment to the patient P as described herein with regard to the treatment module 508.
2. The system 10 may be informed of the patient's medication schedule and may provide reminders to the patients P for when the medications should be taken. The system 10 also may require that the patient P confirm his/her taking of the medication at each scheduled time and may keep a log of this information to assess the patient's ability to manage the medication(s). The system 10 also may help to refill the prescriptions when required.
3. The system 10 also may be informed of the patient's schedule for follow-up healthcare appointments and may provide reminders to the patients P when the appointments are nearing. The system 10 also may require that the patients P confirm their attending of such appointments (e.g., may require the doctor to confirm) and may keep a log of this information to assess the patient's attendance success rate.
4. Based at least in part on the data received in (1) and/or (2) above (as well as from other sources and/or by machine learning), the system 10 may determine if and/or when a patient P may be deemed as “at risk” (e.g., off their medication and/or missing too many appointments). The system 10 may convey this information to the appropriate healthcare providers who may then intervene to provide direct support to the patients P as necessary.
5. The system 10 also may track the location of a patient P (e.g., via GPS), and upon determining that the patient P is in a location deemed “at risk” or “dangerous”, the system 10 may inform the appropriate healthcare providers and/or law enforcement entity to locate and provide support to the patient P as necessary.

It is understood that the actions described above are meant for demonstration and that the system 10 may perform additional actions, not all of the actions described, and any combinations thereof.

### System Structure

FIG. 10 shows aspects of an exemplary project development system 10 of FIG. 1. As shown, the system 10 and backend system 100 comprises various internal applications 600 and one or more databases 700, described in greater detail below. The internal applications 600 may generally interact with the one or more databases 700 and the data stored therein.

The database(s) 700 may comprise one or more separate or integrated databases, at least some of which may be distributed. The database(s) 700 may be implemented in any manner, and, when made up of more than one database, the various databases need not all be implemented in the same way. It should be appreciated that the system is not limited by the nature or location of database(s) 700 or by the manner in which they are implemented.

Each of the internal applications 600 may provide one or more services via an appropriate interface. Although shown as separate applications 600 for the sake of this description, it is appreciated that some or all of the various applications 600 may be combined. The various applications 600 may be implemented in any manner and need not all be implemented in the same way (e.g., using the same software languages, interfaces, or protocols).

In some embodiments, the applications 600 may include one or more of the following applications 600:
1. Data Acquisition application(s) 602. This application 602 may include the data acquisition module 502 as described herein.
2. Response Formulation application(s) 604. This application 604 may include the response formulation module 504 as described herein.
3. Communication application(s) 606. This application 606 may include the communication module 506 as described herein.
4. Treatment applications(s) 608. This application 608 may include the treatment application 508 as described herein.
5. Training application(s) 610. This application 610 may include the training module 510 as described herein.
6. Machine Learning applications 612. This application 612 may include the machine learning module 512 as described herein.

The applications 600 also may include other applications and/or auxiliary applications (not shown). Those of ordinary skill in the art will appreciate and understand, upon reading this description, that the above list of applications is meant for demonstration and that the system 10 may include other applications that may be necessary for the system 10 to generally perform its functionalities as described in this specification. In addition, as should be appreciated, embodiments or implementations of the system 10 need not include all of the applications listed, and that some or all of the applications may be optional. It is also understood that the scope of the system 10 is not limited in any way by the applications that it may include.

In some embodiments, the database(s) 700 may include one or more of the following databases:
1. Incident Information database(s) 702. This database 702 may store any data and/or other types of information related to any incident that may involve use of the system 10.
2. Response Information database(s) 704. This database 704 may store any data, information, and/or assets relating to any response formulated by the system 10 (e.g., via the response formulation application 604) and/or otherwise involved with the system 10.
3. Treatment Information database(s) 706. This database 706 may store any data, information, and/or assets related to any treatments formulated and/or administered by the system 10 or otherwise involved with any incident.
4. Training Information database(s) 708. This database 708 may store any data, information, and/or assets relating to any training simulation(s) and/or materials created and/or used by the system 10.
5. Machine Learning Database(s) 710. This database 710 may store any models (e.g., response model 514, treatment model 516) data, information, and/or assets relating to any machine learning performed by the system 10.

It is understood that the above list of databases is meant for demonstration and that the system 10 may include some or all of the databases, and also may include additional databases as required. It is also understood that the scope of the system 10 is not limited in any way by the databases that it may include.

Various applications 600 and databases 700 in project development system 10 may be accessible via interface(s) 106. These interfaces 106 may be provided in the form of APIs or the like and made accessible to users Un via one or more gateways and interfaces 108 (e.g., via a web-based application 200 and/or a mobile application 200 running on a user's device 300).

It is understood that any aspect and/or element of any of the embodiments described herein or otherwise may be combined in any way to form new embodiments easily understood by a person of ordinary skill in the art and all within the scope of the system 10. Those of ordinary skill in the art will appreciate and understand, upon reading this description, that embodiments hereof may provide different and/or other advantages, and that not all embodiments or implementations need have all advantages.

### Computing

The services, mechanisms, operations, and acts shown and described above are implemented, at least in part, by software running on one or more computers or computer systems or devices. It should be appreciated that each user device is, or comprises, a computer system.

Programs that implement such methods (as well as other types of data) may be stored and transmitted using a variety of media (e.g., computer readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general purpose computers, special purpose computers and computing devices. One or more such computers or computing devices may be referred to as a computer system.

FIG. 5 is a schematic diagram of a computer system 800 upon which embodiments of the present disclosure may be implemented and carried out.

According to the present example, the computer system 800 includes a bus 802 (i.e., interconnect), one or more processors 804, one or more communications ports 814, a main memory 806, removable storage media 810, read-only memory 808, and a mass storage 812. Communication port(s) 814 may be connected to one or more networks by way of which the computer system 800 may receive and/or transmit data.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 804 can be (or include) any known processor, such as, but not limited to, an Intel® Itanium® or Itanium 2® processor(s), AMD® Opteron® or Athlon MP® processor(s), or Motorola® lines of processors, and the like. Communications port(s) 814 can be any of an RS-232 port for use with a modem-based dial-up connection, a 10/100 Ethernet port, a Gigabit port using copper or fiber, or a USB port, and the like. Communications port(s) 814 may be chosen depending on a network such as a Local Area Network (LAN), a Wide Area Network (WAN), a CDN, or any network to which the computer system 800 connects. The computer system 800 may be in communication with peripheral devices (e.g., display screen 816, input device(s) 818) via Input/Output (I/O) port 820. Some or all of the peripheral devices may be integrated into the computer system 800, and the input device(s) 818 may be integrated into the display screen 816 (e.g., in the case of a touch screen).

Main memory 806 can be Random Access Memory (RAM), or any other dynamic storage device(s) commonly known in the art. Read-only memory 808 can be any static storage device(s) such as Programmable Read-Only Memory (PROM) chips for storing static information such as instructions for processor(s) 804. Mass storage 812 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 802 communicatively couples processor(s) 804 with the other memory, storage and communications blocks. Bus 802 can be a PCI/PCI-X, SCSI, a Universal Serial Bus (USB) based system bus (or other) depending on the storage devices used, and the like. Removable storage media 810 can be any kind of external hard-drives, floppy drives, IOMEGA® Zip Drives, Compact Disc-Read Only Memory (CD-ROM), Compact Disc-Re-Writable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor, or a like device. Such a medium may take many forms, including but not limited to, non-volatile media, volatile media, and transmission media. Non-volatile media include, for example, optical or magnetic disks and other persistent memory. Volatile media include dynamic random-access memory, which typically constitutes the main memory of the computer. Transmission media include coaxial cables, copper wire and fiber optics, including the wires that comprise a system bus coupled to the processor. Transmission media may include or convey acoustic waves, light waves, and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer readable media may be involved in carrying data (e.g., sequences of instructions) to a processor. For example, data may be (i) delivered from RAM to a processor; (ii) carried over a wireless transmission medium; (iii) formatted and/or transmitted according to numerous formats, standards, or protocols; and/or (iv) encrypted in any of a variety of ways well known in the art.

A computer-readable medium can store (in any appropriate format) those program elements that are appropriate to perform the methods.

As shown, main memory 806 is encoded with application(s) 822 that support(s) the functionality as discussed herein (an application 822 may be an application that provides some or all of the functionality of one or more of the mechanisms described herein). Application(s) 822 (and/or other resources as described herein) can be embodied as software code such as data and/or logic instructions (e.g., code stored in the memory or on another computer readable medium such as a disk) that supports processing functionality according to different embodiments described herein.

During operation of one embodiment, processor(s) 804 accesses main memory 806 via the use of bus 802 in order to launch, run, execute, interpret or otherwise perform the logic instructions of the application(s) 822. Execution of application(s) 822 produces processing functionality of the service(s) or mechanism(s) related to the application(s). In other words, the process(es) 824 represents one or more portions of the application(s) 822 performing within or upon the processor(s) 804 in the computer system 800.

It should be noted that, in addition to the process(es) 824 that carries(carry) out operations as discussed herein, other embodiments herein include the application 822 itself (i.e., the unexecuted or non-performing logic instructions and/or data). The application 822 may be stored on a computer readable medium (e.g., a repository) such as a disk or in an optical medium. According to other embodiments, the application 822 can also be stored in a memory type system such as in firmware, read only memory (ROM), or, as in this example, as executable code within the main memory 806 (e.g., within Random Access Memory or RAM). For example, application 822 may also be stored in removable storage media 810, read-only memory 808, and/or mass storage device 812.

Those skilled in the art will understand that the computer system 800 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware, or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

As used in this description, the term “portion” means some or all. So, for example, “A portion of X” may include some of “X” or all of “X”. In the context of a conversation, the term “portion” means some or all of the conversation.

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

As used herein, including in the claims, the phrase “using” means “using at least,” and is not exclusive. Thus, e.g., the phrase “using X” means “using at least X.” unless specifically stated by use of the word “only”, the phrase “using X” does not mean “using only X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram the activities associated with those boxes may be performed in any order, including fully or partially in parallel.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of managing an encounter between a first user and a second user and of administrating a medical treatment to the first user based at least in part on the encounter, the method comprising:
(A) by one or more computer systems, receiving first information related to the first user and an incident;
(B) by one or more computer systems, based at least in part on the first information received in (A), deploying a second user to a location of the incident;
(C) by a mobile application, providing the second user with at least a portion of the first information received in (A);
(D) by one or more computer systems, based at least in part on the first information, formulating a first response pertaining to the incident;
(E) by the mobile application, providing second information based at least in part on the first response to the second user;
(F) implementing the first response;
(G) by the mobile application, receiving third information from the second user based at least in part on an outcome of the implementation of the first response;
(H) by one or more computer systems, based at least in part on the first information, the second information, and/or the third information, determining a mental health disorder pertaining to the first user;
(I) by one or more computer systems, based at least in part on the first information, the second information, the third information, and the mental health disorder determined in (H), determining a medical treatment for the first user to alleviate the mental health disorder;
(J) administrating the medical treatment determined in (I) to the first user to alleviate the mental health disorder;
wherein the mental health disorder includes depression, and the medical treatment includes psychotherapy.

2. The method of claim 1 wherein the psychotherapy includes at least one of cognitive behavioral therapy, dialectic behavioral therapy, psychodynamic therapy, and interpersonal therapy

3. The method of claim 1 wherein the medical treatment further includes administering an antidepressant medication to the first user chosen from the group: a selective serotonin reuptake inhibitor (SSRI), and a selective serotonin and norepinephrine inhibitor (SNRI).

4. The method of claim 1 wherein the mental health disorder further includes substance addiction, and the medical treatment further includes detox.

5. The method of claim 1 wherein the formulating of the first response in (D) includes using machine learning including a neural network.

6. The method of claim 1 wherein the determining of the mental health disorder pertaining to the first user in (H) includes using machine learning including a neural network.

7. The method of claim 1 wherein the determining a medical treatment for the first user in (I) includes using machine learning including a neural network.

8. The method of claim 1 wherein the first information includes at least one of first user medical history information, first user criminal history information, and incident background information.

9. The method of claim 1 wherein the first response includes guidance to deescalate the incident.

10. A method of managing an encounter between a first user and a second user and of administrating a medical treatment to the first user based at least in part on the encounter, the method comprising:
(A) by one or more computer systems, receiving first information related to the first user and an incident;
(B) by one or more computer systems, based at least in part on the first information received in (A), deploying a second user to a location of the incident;
(C) by a mobile application, providing the second user with at least a portion of the first information received in (A);
(D) by one or more computer systems, based at least in part on the first information, using first machine learning including a first neural network to formulate a first response pertaining to the incident;
(E) by the mobile application, providing second information based at least in part on the first response to the second user;
(F) implementing the first response;
(G) by the mobile application, receiving third information from the second user based at least in part on an outcome of the implementation of the first response;
(H) by one or more computer systems, based at least in part on the first information, the second information, and/or the third information, determining a mental health disorder pertaining to the first user;
(I) by one or more computer systems, based at least in part on the first information, the second information, the third information, and the mental health disorder determined in (H), determining a medical treatment for the first user to alleviate the mental health disorder;
(J) administrating the medical treatment determined in (I) to the first user to alleviate the mental health disorder.

11. The method of claim 10 wherein the mental health disorder includes depression, and the medical treatment includes psychotherapy.

12. The method of claim 10 wherein the psychotherapy includes at least one of cognitive behavioral therapy, dialectic behavioral therapy, psychodynamic therapy, and interpersonal therapy

13. The method of claim 10 wherein the medical treatment further includes administering an antidepressant medication to the first user chosen from the group: a selective serotonin reuptake inhibitor (SSRI), and a selective serotonin and norepinephrine inhibitor (SNRI).

14. The method of claim 10 wherein the mental health disorder further includes substance addiction, and the medical treatment further includes detox.

15. The method of claim 10 wherein the determining of the mental health disorder pertaining to the first user in (H) includes using machine learning including a neural network.

16. The method of claim 10 wherein the determining a medical treatment for the first user in (I) includes using machine learning including a neural network.

17. The method of claim 10 wherein the first information includes at least one of first user medical history information, first user criminal history information, and incident background information.

18. The method of claim 10 wherein the first response includes guidance to deescalate the incident.
